(19) United States
(12) Patent Application Publication
(10) Pub. No.: US 2014/0004522 A1
(43) Pub. Date: Jan. 2, 2014

(54) Method for Detecting a Target Analyte Using a Self-Cleaving Synthetic Molecule

(71) Applicant: QURA Scientific, LLC, (US)

(72) Inventor: Heath Bowers, Parma Heights, OH (US)

(73) Assignee: QURA Scientific, LLC, Parma Heights, OH (US)

(21) Appl. No.: 13/674,366

(22) Filed: Nov. 12, 2012

Related U.S. Application Data
(60) Provisional application No. 61/558,481, filed on Nov. 11, 2011.

(51) Int. Cl. G01N 33/53; G01N 33/574

(52) U.S. Cl. CPC G01N 33/53; G01N 33/57496
USPC 435/6.12

(57) ABSTRACT

One aspect of the present disclosure relates to a method for determining the amount of a target analyte in a sample. One step of the method can include contacting the sample with a self-cleaving synthetic molecule to form a first treated sample. The self-cleaving synthetic molecule can include at least one target analyte-specific aptamer. Next, the first treated sample can be contacted with at least one actuating molecule to form a second treated sample. The second treated sample is then subjected to a target amplification assay. A detectable signal is produced by the target amplification assay when the target analyte is present in the sample.

### RELATED APPLICATION

This application claims priority to U.S. Provisional Application. No. 61/558,481, filed Nov. 11, 2011, which is incorporated herein by reference.

### TECHNICAL FIELD

The present disclosure relates generally to methods for detecting analytes in various samples and, more particularly, to methods for quantifying analytes in various samples using self-cleaving synthetic molecules and target amplification assays.

### BACKGROUND

With the advent of molecular diagnostics, personalized medicine, and pathogen identification, there is a need for a dependable, sensitive, and reproducible method that detects proteins and small molecules. Reliable and rapid identification of biomarkers from patient or biological sources is required to ensure a proper and timely diagnosis. Limitations for current assay platforms include laborious sample purification needed to achieve target enrichment, design and expense of specific signal-producing molecules, and the inability to collect reliable, reproducible, and quantitative data. Additionally, the low abundance of many clinically-relevant biomarkers in samples renders them impractical for detection using conventional methods.

### SUMMARY

The present invention relates generally to methods for detecting analytes in various samples and, more particularly, to methods for quantifying analytes in various samples using self-cleaving synthetic molecules and target amplification assays.

One aspect of the present disclosure relates to a method for determining the amount of a target analyte in a sample. One step of the method can include contacting the sample with a self-cleaving synthetic molecule to form a first treated sample. The self-cleaving synthetic molecule can include at least one target analyte-specific aptamer. Next, the first treated sample can be contacted with at least one actuating molecule to form a second treated sample. The second treated sample can then be subjected to a target amplification assay. A detectable signal can be produced by the target amplification assay when the target analyte is present in the sample.

Another aspect of the present disclosure relates to a method for determining the amount of a target polypeptide in a sample. One step of the method can include contacting the sample with a type I hammerhead ribozyme to form a first treated sample. The type I hammerhead ribozyme can include at least one target analyte-specific aptamer and first and second primer sites at the 3′ and 5′ ends thereof, respectively. Next, the first treated sample can be contacted with at least one actuating molecule to form a second treated sample. The second treated sample can then be subjected to a reverse transcriptase-quantitative polymerase chain reaction. The 3′ primer site is not cleaved from the type 1 hammerhead ribozyme in the presence of the target analyte. An uncleaved target analyte-specific aptamer is amplified by the target amplification assay to produce a detectable signal.

Another aspect of the present disclosure relates to a kit for determining the amount of a target analyte in a sample. In some instances, the kit can include at least one test tube for preparing a sample for a target amplification reaction, reagents for conducting the method of the present disclosure, and instructions for detecting the target analyte according to the method of the present disclosure.

### BRIEF DESCRIPTION OF THE FIGURES

FIG. 1 is a process flow diagram illustrating a method for determining the amount of a target analyte in a sample according to one aspect of the present disclosure.

FIGS. 2A-B are schematic illustrations depicting one example of a self-cleaving synthetic molecule (FIG. 2A), as well as 5′ and 3′ primer binding sites (shaded regions) (FIG. 2B) and a 3′ cleavage site (indicated by arrow) thereof.

FIGS. 3A-B are schematic illustrations depicting a self-cleaving synthetic molecule in the absence (FIG. 3A) and presence (FIG. 3B) of a target analyte.

### DETAILED DESCRIPTION

It should be understood that the present disclosure is not limited to the particular methodology, protocols, and reagents, etc., described herein and, as such, may vary. The terminology used herein is for the purpose of describing particular aspects only, and is not intended to limit the scope of the present disclosure, which is defined solely by the claims.

The section headings are used herein for organizational purposes only and are not to be construed as in any way limiting the subject matter described.

The methods and techniques of the present disclosure are generally performed according to conventional methods well-known in the art, and as described in various general and more specific references that are cited and discussed throughout the present disclosure unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

As used herein, the terms below are defined by the following meanings.

“A” or “an” as used herein can mean one or more than one, such as at least one. Where the plural form is used herein, it generally includes the singular.

“Comprising” can mean, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of is a synonym of “comprising” (see above).

The terms “analyte” or “ligand” (or grammatical equivalents) as used herein can refer to any molecule or compound to be detected by the present disclosure, and that can interact with a target analyte-specific aptamer to be designed and/or selected as described herein. Suitable ligands or analytes can include, but are not limited to, small chemical molecules, such as environmental or clinical chemicals, pollutants or biomolecules including, but not limited to, pesticides, insecticides, toxins, therapeutic and abused drugs, hormones, antibiotics, antibodies, organic materials, etc. Suitable biomolecules can include, but are not limited to, proteins (including enzymes, immunoglobulins and glycoproteins), nucleic acids, lipids, lectins, carbohydrates, hormones, whole cells (including prokaryotic, such as pathogenic bacteria) and eukaryotic cells, including mammalian tumor cells, viruses, spores, etc. Illustrative analytes that are proteins can include, but are not limited to, enzymes, drugs, cells, antibodies, antigens, cellular membrane antigens and receptors (neural, hormonal, nutrient, and cell surface receptors), or their natural ligands.

The term “self-cleaving synthetic molecule” as used herein can refer to any synthetic molecule that includes at least one target analyte-specific aptamer and is capable of self-cleavage in the presence or absence of a target analyte.

The term “actuating molecule” as used herein can refer to any agent, compound, or substance that is capable of directly or indirectly causing cleavage of a self-cleaving synthetic molecule. Exemplary actuating molecules can include, but are not limited to, inorganic molecules (e.g., magnesium, sodium, potassium, manganese and calcium) and organic molecules (e.g., nucleoside triphosphates, amino acids, nucleic acids, sugars and fatty acids).

The term “target amplification assay” as used herein can refer to any assay, technique, or procedure capable of increasing the number of copies of a target molecule (e.g., a nucleic acid) from a sample containing an initial number (e.g., lower) of copies of the target molecule. In one example, a target amplification assay is capable of exponentially increasing the number of copies of a target molecule.

The terms “nucleotide” or “nucleic acid” as used herein can refer to naturally- and non-naturally-occurring nucleotides and nucleotide analogs. Nucleotides can include, but are not limited to, adenosine, cytosine, guanosine, thymidine, uracil, 4-acetylcytosine, 8-hydroxy-N6-methyladenosine, aziridinyl-cytosine, pseudoisocytosine, 5-(carboxyhydroxylmethyl)uracil, 5 -fluorouracil, 5-bromouracil, 5-carboxymethylaminomethyl-2-thiouracil, 5 -carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine and 2,6-diaminopurine. Additionally, the terms “nucleic acid sequence,” “nucleic acid molecule,” and “polynucleotide” as used herein can refer to a DNA sequence or analog thereof, an RNA sequence or analog thereof, or PNA sequence or analog thereof. Nucleic acids are formed from nucleotides including, but not limited to, the nucleotides listed above.

The term “hammerhead ribozyme” as used herein can refer to a ribozyme that contains a core, three stems that extend from the core, referred to herein as stem I, stem II and stem III, and at least one loop, which is located on the opposite end of a stem from the core. In instances where the ribozyme is a trans-acting type I ribozyme, it can contain one loop, e.g., at the end of stem II (referred to as loop II). In other instances where the ribozyme is a cis-acting type I ribozyme, the ribozyme can contain two loops, one located at the end of stem II (referred to as loop II) and the other located at the end of stem III (referred to as loop III).

The term “cis-cleaving hammerhead ribozyme” as used herein can refer to a hammerhead ribozyme that, prior to cleavage, is comprised of a single polynucleotide. A cis-cleaving hammerhead ribozyme is capable of cleaving itself.

The term “trans-cleaving hammerhead ribozyme” as used herein can refer to a hammerhead ribozyme that, prior to cleavage, is comprised of at least two polynucleotides. One of the polynucleotides can be the target sequence that is cleaved.

The term “complementary” as used herein can refer to a nucleotide or nucleotide sequence that hybridizes to a given nucleotide or nucleotide sequence. For instance, for DNA, the nucleotide A is complementary to T and vice versa, and the nucleotide C is complementary to G and vice versa. For instance, in RNA, the nucleotide A is complementary to the nucleotide U and vice versa, and the nucleotide C is complementary to the nucleotide G and vice versa. Complementary nucleotides include those that undergo Watson-Crick base pairing and those that base pair in alternative modes. For instance, as used herein for RNA, the nucleotide G is complementary to the nucleotide U and vice versa, and the nucleotide A is complementary to the nucleotide G and vice versa. Therefore, in an RNA molecule, the complementary base pairs include A and U, G and C, G and U, and A and G. Other combinations, e.g., A and C or C and U, are considered to be non-complementary base pairs. A complementary sequence can be comprised of individual nucleotides that are complementary to the individual nucleotides of a given sequence, where the complementary nucleotides are ordered such that they will pair sequentially with the nucleotides of the given sequence.

The term “stem” as used herein can refer to a nucleic acid motif that extends from a ribozyme core, at least a portion of which is double-stranded. In certain aspects, there is a loop at the opposite end of the stem from the ribozyme core, and this loop connects the two strands of the double-stranded stem. In other aspects, a stem can comprise about 2 to about 20 complementary base pairs. In further aspects, a stem can comprise about 3, 4, 5, 6, 7, 8, or 9 complementary base pairs.

In certain aspects, at least 30% of the nucleotides in a stem are part of a complementary base pair. The remaining base pairs may be mismatched, non-complementary base pairs, or may be part of a bulge. In certain aspects, at least 40% of the nucleotides in a stem are part of a complementary base pair. In certain aspects, at least 50% of the nucleotides in a stem are part of a complementary base pair. In certain aspects, at least 60% of the nucleotides in a stem are part of a complementary base pair. In certain aspects, at least 70% of the nucleotides in a stern are part of a complementary base pair. In certain aspects, at least 80% of the nucleotides in a stem are part of a complementary base pair. In certain aspects, at least 90% of the nucleotides in a stem are part of a complementary base pair. In certain aspects, at least 95% of the nucleotides in a stem are part of a complementary base pair. In certain aspects, at least 99% of the nucleotides in a stem are part of a complementary base pair. In certain aspects, 100% of the nucleotides in a stem are part of a complementary base pair.

The term “loop” as used herein can refer to a sequence of nucleotides that is not paired with another strand and is located at the distal end of a stem that is opposite the core. In certain aspects, a loop is between about 1 to 20 nucleotides long. In other aspects, a loop is between about 2 and 10 nucleotides long. In further aspects, a loop is between about 3 and 8 nucleotides long. A loop is numbered according to the stem to which it is attached. Therefore, loop I is located at the end of stem I opposite the core, loop II is located at the end of stem II opposite the core, and loop III is located at the end of stem III opposite the core.

The term “polypeptide” as used herein can refer to an oligopeptide, peptide or protein, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term can also include peptides and polypeptide fragments, motifs and the like, glycosylated polypeptides, and all mimetic and peptidomimetic polypeptide forms.

### Overview

Methods for antibody-based detection of proteins and small molecules are well known and have been the predominant method utilized since their inception in 1979. Western blotting, for example, entails protein separation on a polyacrylamide gel with or without sodium dodecyl sulfate. Protein is then transferred to a membrane and probed with anti-serum against targets of interest. The antibodies within the anti-serum bind the proteins of interest while the remaining anti-serum is washed away. The antibodies from the anti-serum are then detected with radioiodinated protein A and radiography film.

Since the development of Western blots, many improvements have been made to the procedure. Today, Western blots typically use purified antibodies that may be conjugated to horseradish peroxidase, alkaline phosphatase, or fluorescent and infrared moieties, which eliminate the need for radioactivity and increase sensitivity. The method by which protein is transferred to a membrane has also been optimized, with passive transfer replaced by electroblotting to reduce procedure time. Stronger membranes, such as nitrocellulose or polyvinylidene difluoride are easy to handle and exhibit high protein binding and low background signal for improved signal-to-noise ratio. Additionally, a number of products have been developed for Western blotting that reduce the time of individual steps in the procedure or that fully automate the process.

Despite the improvements made to Western blotting, there are still several shortcomings associated with the technique. For example, antibodies used in Western blotting may require three to six months for production in animals or animal-derived cells. Additionally, the process requires a large monetary and material investment, as a significant amount of purified protein or peptide must be supplied to elicit an immune response and validate the antibody. There is also no guarantee that a protein or peptide will generate a sufficient immune response. Furthermore, Western blotting is laborious as it can take a minimum of six to twenty-four hours to complete by conventional methods. There are numerous steps in the process where variability or error can be introduced. While there are some commercially available products that reduce the number of blotting steps or blotting time, there is a trade-off in sensitivity and signal-to-noise ratio. Often, the results obtained from Western blots are not quantitatively reproducible, which allows for qualitative interpretations only.

Much like Western blots, enzyme-linked immunosorbent assays (ELISAs) utilize antibodies for protein detection and have been in use since the 1970's. An ELISA typically entails a procedure where an antibody specific for a target is covalently tethered to a support surface, such as cellulose. The sample containing an alkaline phosphatase-conjugated target is then added to the tethered antibody so that the target is “captured” on the surface. After washing, the alkaline phosphatase substrate pNPP is added and a color change is observed, which is dependent on the amount of target bound to antibody. ELISAs are frequently used in diagnostic assays and for screening assays in the food and drug industries because they are more convenient than Western blots, and the results are quantifiable. Modern ELISAs have evolved to use reporters that increase sensitivity, and in some cases allow multiplexing of targets. Detection is achieved by enzymatic or fluorogenic reporters or by qPCR. ELISAs, however, are multi-step processes that may require multiple antibodies per target molecule and, thus, can be very time-consuming.

A new and growing field for protein detection utilizes nucleic acid aptamers to detect proteins and small molecules of interest. Although these approaches eliminate current problems associated with antibodies, these methods introduce new disadvantages. Aptamer-quantum dot conjugates, for example, cannot be used in high throughput applications and are still time-consuming as only a limited number of samples can be tested since protein separation and transfer are still required. Protection assays using aptamers and DNAse I have also been developed; however, such assays yield only qualitative results and suffer from obvious risks associated with adding DNase to an assay that is based on DNA amplification. Despite the use of a ribozyme-aptamer complex for protein detection, the use of a FRET dye pair limits the usefulness of this detection method. The dyes required for FRET generally photobleach quickly and decay over time, which does not allow for long term storage or repeat use.

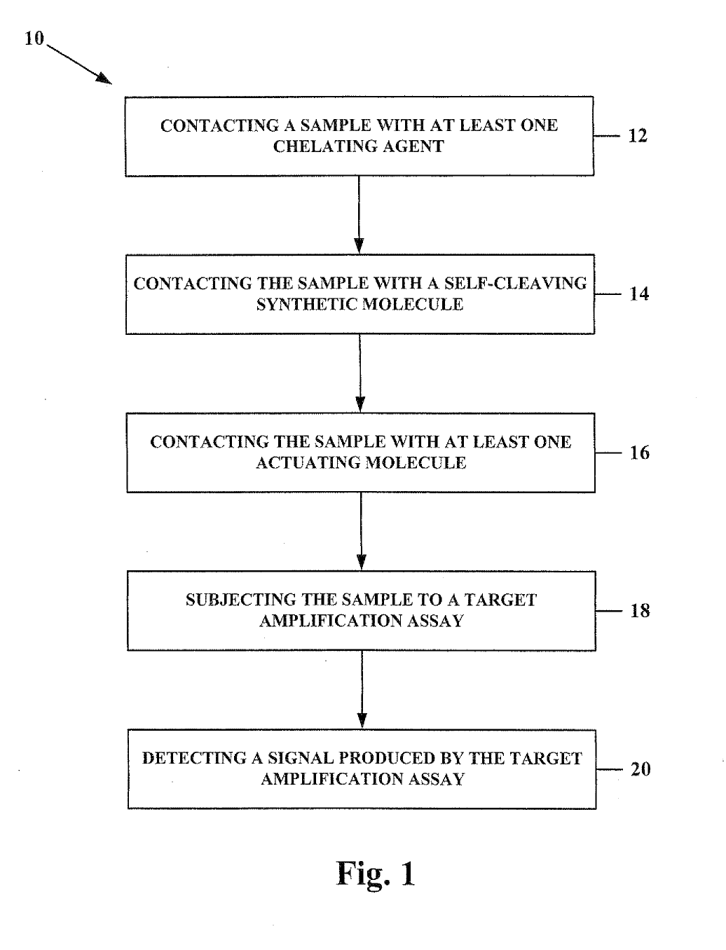

As shown in FIG. 1, one aspect of the present disclosure includes a method 10 for target analyte detection that results in quantitative, reproducible data that is more sensitive and reliable than current methods of analyte detection (e.g., polypeptide and small molecule). The present disclosure is generally predicated on the notion that target amplification assays, such as quantitative polymerase chain reaction (qPCR) can replace Western blotting for analyte detection just as Northern and Southern blotting have been replaced, ultimately yielding highly reproducible and quantitative data in a shorter and more streamlined process. The highly sensitive method 10 of the present disclosure is capable of generating a robust detection signal against a single target analyte or epitope site due to the ability of target amplification assays to exponentially increase copies of a target in a given sample. Advantageously, the method 10 surpasses the sensitivity limits of current protein detection technologies and can be performed in a single tube without isolation of the target analyte from the sample. Further, the method 10 can be completed in as little as about 2 hours, which is significantly less time than comparable Western blotting or ELISAs.

### Exemplary Aspects

One aspect of the present disclosure includes a method 10 (FIG. 1) for detecting the amount of a target analyte in a sample. Examples of target analytes detectable by the method 10 are provided above. In one example, the method 10 can be used to detect the amount of a target polypeptide or protein in a sample. In another example, the method 10 can be used to detect the amount of a small molecule (e.g., a ligand having a molecular weight of less than about 2500 amu) in a sample.

The sample can include any one or combination of material(s) having, or being suspected of having, a target analyte. In some instances, the sample can comprise a biological tissue or fluid. For example, the sample can comprise one or more cells obtained (e.g., by biopsy) from a subject (e.g., a human or non-human subject). Alternatively, the sample can comprise a biological fluid, such as blood, plasma, serum, cerebrospinal fluid, saliva, urine, etc. Advantageously, the sample need not be subjected to a purification or separation step prior to conducting the method 10 in order to isolate the target analyte. This is unlike conventional protein detection methods, such as Western blotting and ELISA in which a target protein is typically purified or separated prior to detection. The ability to forgo a purification or separation step significantly reduces the time needed to complete the method 10, which, in some instances, may take less than about 2 hours, less than about 90 minutes, or less than about 60 minutes to complete. This is in contrast to conventional protein detection assays, which typically require at least 4 hours to complete. The ability to forgo a purification or separation step further reduces the quantity of required reagents, thereby reducing the overall cost associated with the method 10.

At Step 12, the sample can be contacted with at least one chelating agent. The at least one chelating agent can be contacted with the sample for a time and in an amount to prevent or reduce premature cleavage of the self-cleaving synthetic molecule. Examples of suitable chelating agents can include, but are not limited to, ethylenediaminetetraacetic acid (EDTA), nitrilotriacetic Acid (NTA), diethylenetriaminepentaacetic acid (DTPA), and ethylene glycol tetraacetic acid (EGTA). In some instances, the sample can be contacted with the chelating agent(s) in a buffered (e,g., trishydroxymethylaminomethane (TRIS) pH 7-9, 3-morpholinopropanesulfonic acid (MOPS) pH 7-9, Phosphate Buffer pH 7-9, and N-[Tris(hydroxymethyl)methyl]-3-aminopropanesulfonic acid (TAPS) pH 7-9), low salt environment (e.g., a salt concentration between about 20 mM and about 100 mM) at a temperature of about 4° C. to about 32° C. In some instances, one or more non-specific nucleic acids (e.g., tRNA, oligonucleotides, or fish sperm DNA) can be contacted with the sample to prevent or reduce sample degradation from endogenous nucleases. Non-specific nucleic acids can be contacted with the sample either before, concurrent with, or after contacting the sample with the chelating agent(s).

At Step 14, the sample can be contacted with a self-cleaving synthetic molecule. The self-cleaving synthetic molecule is contacted with the sample for a time and in an amount sufficient for the at least one target analyte-specific aptamer to bind to the target analyte, if present in the sample. It will be appreciated, however, that the sample may, in some instances, not be contacted with a chelating agent (or agents) prior to Step 14. The self-cleaving synthetic molecule can include at least one target analyte-specific aptamer that is capable of selectively binding a target analyte. The choice of target analyte to which the at least one target analyte-specific aptamer (and thus the self-cleaving synthetic molecule) binds is vast. For example, target analytes can be naturally or non-naturally occurring molecules, including peptides, small organic molecules (including drugs and certain metabolites and intermediates, cofactors, etc), and metal ions. Exemplary analytes that can bind to an analyte-specific aptamer can include those listed above, as well as drugs, metabolites, intermediates, cofactors, transition state analogs, ions, metals, nucleic acids, and toxins. Analyte-specific aptamers may also bind natural and synthetic polymers, including proteins, peptides, nucleic acids, polysaccharides, glycoproteins, hormones, receptors and cell surfaces, such as cell walls and cell membranes.

An analyte-specific aptamer can be obtained by in vitro selection for binding to a target analyte. However, in vivo selection of an analyte-specific aptamer is also possible. Analyte-specific aptamers have specific binding regions, which are capable of forming complexes with an intended target analyte in an environment, wherein other substances in the same environment are not complexed to the aptamer. The specificity of the binding is defined in terms of the comparative dissociation constants (K_{d}) of the aptamer for its ligand as compared to the dissociation constant of the aptamer for other materials in the environment (or unrelated molecules in general). In some instances, a target analyte can be a ligand which binds to the analyte-specific aptamer with greater affinity than to unrelated material. In other instances, the K_{d} for the analyte-specific aptamer with respect to its analyte can be at least about 10-fold less than the K_{d} for the analyte-specific aptamer with unrelated material or accompanying material in the environment. For example, the K_{d} can be at least about 50-fold less, at least about 100-fold less, or at least about 200-fold less. An analyte-specific aptamer can be between about 10 and about 300 nucleotides in length and, in one example, between about 30 and about 100 nucleotides in length.

Analyte-specific aptamers can be developed to bind target analytes by employing known in vivo or in vitro selection techniques, such as SELEX (Ellington et al., Nature 346, 818-22 (1990); and Tuerk et al., Science 249, 505-10 (1990)). Methods of making aptamers are also described in, for example, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Generally, in their most basic form, in vitro selection techniques for identifying target analyte-specific aptamers involve first preparing a large pool of oligonucleotides of the desired length that contains at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques, although any means that will allow faithful, efficient amplification of selected nucleic acid sequences can be employed. The DNA pool is then transcribed in vitro to produce RNA transcripts. The RNA transcripts may then be subjected to affinity chromatography; although, any protocol that will allow selection of nucleic acids based on their ability to bind specifically to another molecule (e.g., a protein or any target analyte) may be used. In the case of affinity chromatography, the transcripts are most typically passed through a column or contacted with magnetic beads (or the like) on which the target analyte has been immobilized. RNA molecules in the pool, which bind to the ligand are retained on the column or bead while nonbinding sequences are washed away. The RNA molecules that bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules, which are capable of acting as aptamers for the target analyte. Once an aptamer sequence has been successfully identified, the aptarner may be further optimized by performing additional rounds of selection starting from a pool of oligonucleotides comprising the mutagenized aptamer sequence. In one example, the aptamer can be selected for analyte binding in the presence of salt concentrations and temperatures that mimic normal physiological conditions.

One can generally choose a suitable target analyte without reference to whether an aptamer is yet available. In most cases, an aptamer can be obtained that binds the ligand of choice by someone of ordinary skill in the art. The unique nature of the in vitro selection process allows for the isolation of a suitable aptamer that binds a desired analyte despite a complete dearth of prior knowledge as to what type of structure might bind the desired analyte.

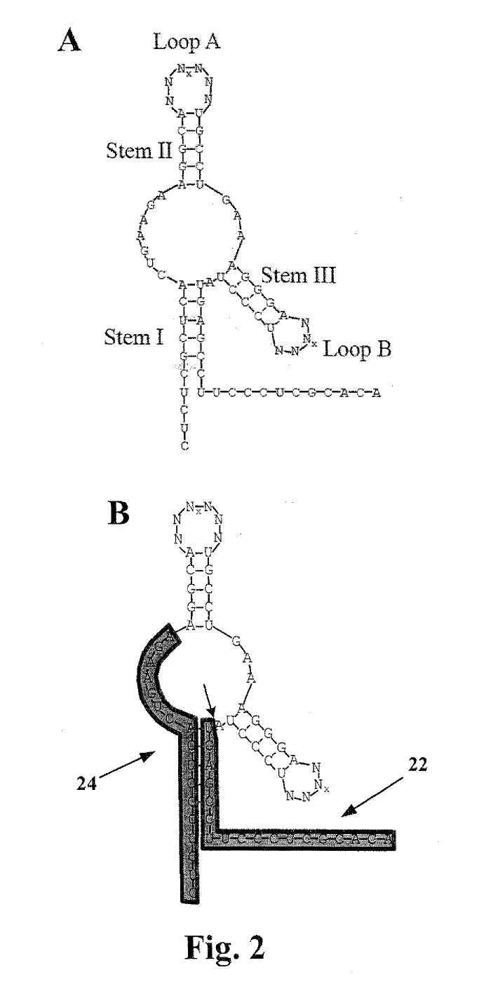

In some instances, the self-cleaving synthetic molecule can include a type hammerhead ribozyme comprising at least one target analyte-specific aptamer. As shown in FIGS. 2A-B, for example, a type I hammerhead ribozyme can comprise a catalytic core and stem I, stem II, and stem III duplex regions extending therefrom. In some instances, each of stem I, stem II, and stem III can further include a single-stranded loop region. As shown in FIG. 2A, stem II and stem III can include single-stranded loop regions designated Loop A and Loop B, respectively. In some instances, the loop region of stem III (Loop B) can include a target analyte-specific aptamer. In other instances, the loop region of stem II (Loop A) can include a target analyte-specific aptamer. In further instances, the loop region of stem II (Loop A) and the loop region of stem III (Loop B) can each include a target analyte-specific aptamer, which is capable of binding the same or different target analyte. Referring to FIG. 2B, the type I hammerhead ribozyme can further include a 3′ primer site 22 (shaded region), a 5′ primer site 24 (shaded region), and a 3′ self-cleavage site (indicated by arrow) that, upon cleavage, results in removal or dissociation of the 3′ primer site 22 from the ribozyme.

In other instances, the self-cleaving synthetic molecule can comprise a type III hammerhead ribozyme, or other similar ribozyme Glm S), and at least one target analyte-specific aptamer so that cleavage of a 5′ cleavage site (not shown) can occur in the presence or absence of a target analyte and results in removal or dissociation of the 5′ primer site from the ribozyme.

In further instances, the self-cleaving synthetic molecule can comprise a PNAzyme or DNAzyme that includes at least one target analyte-specific aptamer directly coupled thereto so that a primer site is released upon cleavage of the self-cleavage site, which may exist anywhere within the PNAzyme or DNAzyme.

At Step 16, the sample (e.g., the first sample) can be contacted with at least one actuating molecule to form a second treated sample. The at least one actuating agent can be added in an amount (e.g., about 1-10 mM), for a time (e.g., about 5-30 minutes), and at a temperature (e.g., about 4-32° C.) sufficient to promote cleavage of the self-cleaving synthetic molecule. In some instances, the at least one actuating molecule can include a cation, such as a metal ion (e.g., a divalent metal ion). Non-limiting examples of metal ions that may be used can include magnesium, calcium, sodium, potassium and aluminum. Alternatively, examples of actuating agents can be organic molecules, such as, but not limited to, triphosphates, amino acids, nucleic acids, sugars and fatty acids.

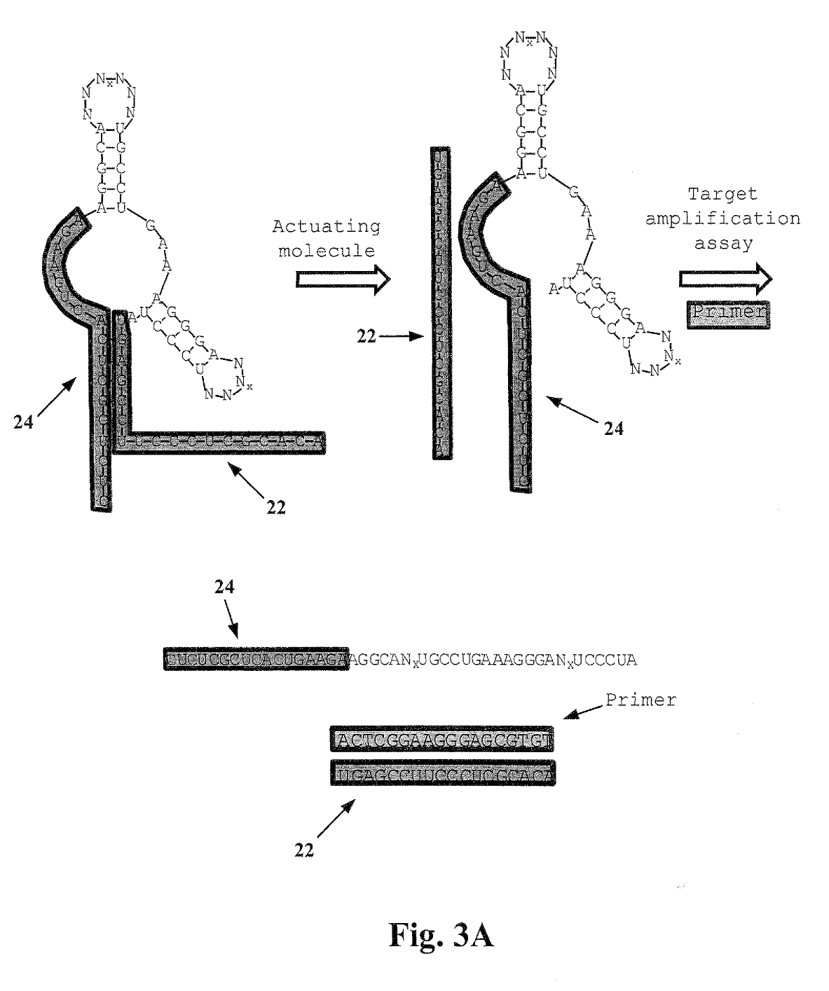

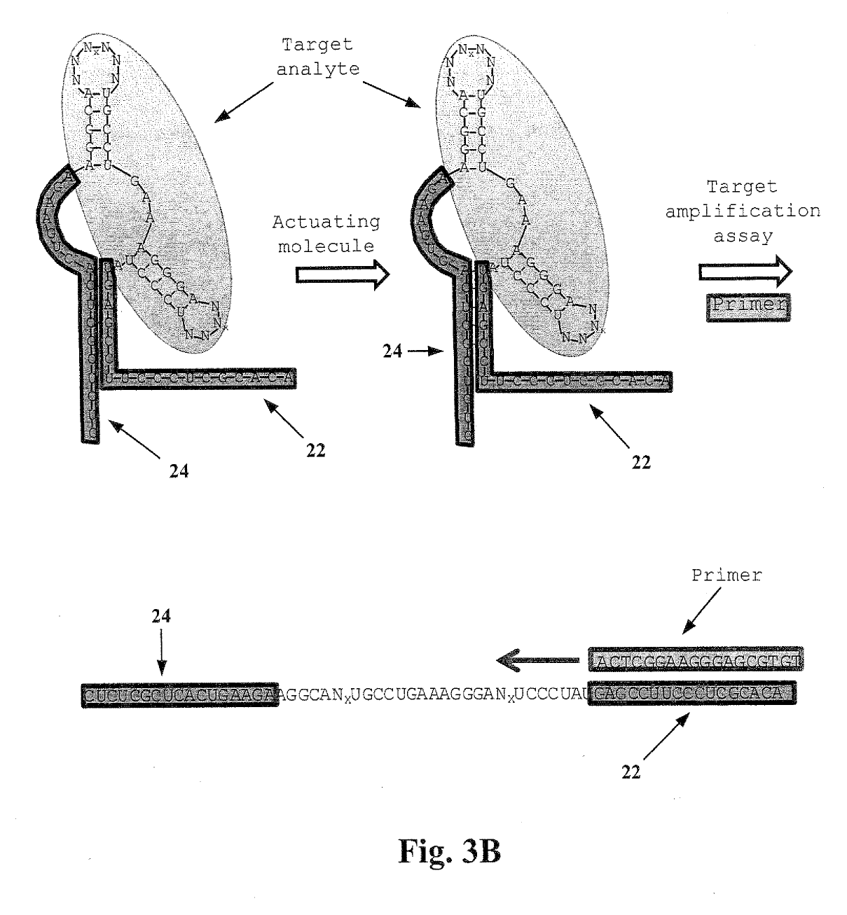

Any target analyte-specific aptamers that are not bound to their respective target analytes are cleaved, thereby removing a primer site required for subsequent amplification and detection (discussed in more detail below). For example, in the absence of target analyte-specific aptamers bound to their respective target analytes, addition of actuating molecules (e.g., cations) removes the primer binding site needed for reverse transcription first strand DNA synthesis, thereby preventing amplification via a target amplification assay (FIG. 3A). Conversely, target analyte-specific aptamers bound to their respective target analytes are protected from cleavage, thereby retaining the primer site required for subsequent amplification and detection (FIG. 3B).

Following the addition of the at least one actuating molecule to the sample, the sample (e.g., the second sample) can be subjected to a target amplification assay (Step 18). Examples of suitable target amplification assays can include qPCR when the self-cleaving synthetic molecule is comprised of DNA and reverse transcriptase qPCR (RT-PCR) when the self-cleaving synthetic molecule is comprised of RNA. In some instances, reagents needed for qPCR amplification of a target-specific aptamer (e.g., bound to its respective target analyte) can be added directly to the sample. Such reagents are known in the art and can include, for example, a thermostable DNA polymerase, a reverse transcriptase, deoxynucleoside triphosphates (dNTPs), primers specific for amplification of the target-specific aptamer, Taqman (Life Technologies, Grand Island, N.Y.), probes, molecular beacons, buffer, or nucleic acid-binding dyes, such as SYBR® (Life Technologies, Grand Island, N.Y.) Green or EvaGreen® (Biotium Hayward, Calif.). The sample can then be placed in a qPCR thermocycler, for example, where amplification can proceed. Protocols for conducting qPCR and RT-PCR are known in the art, such as those disclosed in Bustin S. A. et al., Clinical Chemistry 55, 611-22 (2009).

At Step 20, one or more signals produced by the target amplification assay can be detected and then quantified to determine the amount of the target analyte in the sample. The amount of target analyte present in the sample can be determined, for example, based on obtained cycle threshold (Ct) values from a sample of interest and a standard curve derived from serial dilutions of the self-cleaving synthetic molecule. The method 10 can thus find use in a variety of diagnostic capacities, including protein expression analysis in cells or tissues, viral or pathogen detection, immuno-precipitation, as well as influx of protein into a given system (e.g., by transfection, pathogen production, injection, or any other delivery technique).

In one example of the present disclosure, a method is provided for determining the amount of a target polypeptide in a sample. The sample can include a tissue or fluid sample obtained from a subject (e.g., by biopsy or needle draw). The sample can be placed in a single test tube and then contacted with at least one chelating agent, such as EDTA. For instance, the sample can be contacted with. EDTA in a buffered, low salt environment at a temperature of between about 4° C. and about 32° C. Additionally or optionally, non-specific nucleic acids (e.g., tRNA or fish sperm DNA) can be added to the sample. Next, a self-cleaving type I hammerhead ribozyme including at least one target analyte-specific aptamer, such as the one shown in FIGS. 3A-B can be contacted with the sample to form a first treated sample. In some instances, it may be preferable to use a natural hammerhead ribozyme so that cleavage rates occur quickly due to tertiary interactions between stem I and stem II. The type I hammerhead ribozyme can comprise a catalytic core and stem I, stem II, and stem III duplex regions extending therefrom. Each of stem II and stem III can include a single-stranded loop region. In some instances, it may be preferable to place the target analyte-specific aptamer in the loop of stem III to allow for the interaction between stem I and stem II. The type I hammerhead ribozyme can further include first and second primer sites at the 3′ and 5′ ends thereof (respectively). The self-cleaving type I hammerhead ribozyme is contacted with the sample in an amount and for a time sufficient to allow any target polypeptides present in the sample to bind to the target analyte-specific aptamer(s).

After a sufficient period of time, the first treated sample is contacted with at least one actuating molecule, such as divalent magnesium to form a second treated sample. In the absence of the target polypeptide, addition of divalent magnesium removes the 3′ primer site as a result of self-cleavage of the type I hammerhead ribozyme. Alternatively, in the presence of the target polypeptide, the target polypeptide(s) bind the target analyte-specific aptamer(s) and preserve the 3′ primer site even after addition of divalent magnesium. The sample can then be subjected to RT-qPCR performed in 50 millimolar potassium chloride, 5 percent glycerol, 10 millimolar Tris pH 9.0, 4 millimolar magnesium chloride, 0.2 millimolar deoxynucleoside triphosphates, 200 U of Moloney-Murine Leukemia virus reverse transcriptase (M-MLV RT), 2 U of hot-start thermus aquaticus polymerase (Taq), 20 U of RNase inhibitor, 0.2 micromolar ribozyme-specific primers, and SYBR® Green cycled for 2 stages with the first stage containing a 42° C. step for 30 minutes followed by 10 minutes at 95° C. and the second stage containing a 95° C. step for 10 seconds followed by 1 minute at 60° C. Since the polypeptide-bound aptamer(s) is/are protected from cleavage, the 3′ primer site(s) is/are present and available for primer hybridization and, thus, reverse transcription first strand DNA synthesis. Reverse transcription of the type I hammerhead ribozyme can proceed, resulting in a detectable RT-qPCR signal. The detection of an RT-qPCR signal can then be quantified via Ct values, thereby indicating the amount of the target polypeptide present in the sample.

Another aspect of the present disclosure can include a kit for determining the amount of a target analyte in a sample. The kit can generally include one or more of the reagents required for carrying out the method 10 of the present disclosure, as well as instructions for carrying out the method. Kits comprising various components used in carrying out the method 10 may be configured for use in any procedure requiring amplification of nucleic acid target molecules, and such kits can be customized for various different end-users. Suitable kits may be prepared, for example, for blood screening, disease diagnosis, infection control, environmental analysis, criminal investigations or other forensic analyses, genetic analyses, archeological or sociological analyses, or for general laboratory use. Kits of the present disclosure can provide one or more of the components necessary to carry out nucleic acid amplifications according to the method 10. Kits may include reagents suitable for amplifying nucleic acids from one particular target or may include reagents suitable for amplifying multiple targets. Kits may comprise a carrier that may be compartmentalized to receive in close confinement one or more containers, such as vials, test tubes, wells and the like. In one example, a kit can include a single test tube that may be used to perform the entire method 10.

A kit according to the present disclosure can include, for example, in one or more containers, a chelating agent, a self-cleaving synthetic molecule, an actuating molecule, reagents needed to conduct a target amplification assay, such as potassium chloride, glycerol, Tris, magnesium chloride, deoxynucleoside triphosphates, M-MLV RT, hot-start Taq. RNase inhibitor, ribozyme-specific primers, and SYBR® Green. In some instances, a kit of the present disclosure can also include one or more containers containing one or more positive and negative control target nucleic acids, which can be utilized in amplification experiments to validate test amplifications carried out by an end user. In other instances, one or more of the reagents listed above may be combined with an internal control. It is also possible to combine one or more of these reagents in a single tube or other containers.

Supports suitable for use with kits of the present disclosure (e.g., test tubes, multi-tube units, multi-well plates, cuvettes, flexible containers, microfluidic devices, including analytical cards or discs for use in centrifugal analyzers, etc.) may also be supplied with reagents of the present disclosure. Finally, a kit of the present disclosure may include one or more instruction manuals provided in written or electronic form, including CD-ROMs, DVDs and video tapes. Kits of the present disclosure may contain virtually any combination of the components set out above or described elsewhere herein. As one skilled in the art would recognize, the components supplied with kits of the present disclosure will vary with the intended use for the kits, and the intended end user. Thus, kits may be specifically designed to perform various functions set out in this application and the components of such kits will vary accordingly.

The present disclosure will be further described by reference to the following detailed examples.

### EXAMPLES

### Example 1

### Detection of Hemagglutinin-Tagged Plasminogen Activator Inhibitor-2 (HA-PAI-2) from Co-Immunoprecipitations

For the purpose of Example 1, a type I hammerhead ribozyme containing a self-cleaving hemagglutinin-specific aptamer in the loop of stem III is used.

Mouse embryonic fibroblast (MEF) cells (1×10⁶) stably expressing HA-PAI-2 and human transglutaminase 2 (TG2) are lysed in Radioimmunoprecipitation Assay (RIPA) buffer containing 2 millimolar EDTA. The cellular debris is pelleted at 14,000×g and 4° C. for 10 minutes. The lysate is then incubated with mouse monoclonal antibodies against TG2 or mouse IgG (control) at 4° C. for 2 hours. Next, the lysate is incubated with pre-equilibrated beads conjugated to rabbit anti-mouse IgG at 4° C. overnight (roughly 16 hours). The beads are pelleted at 3,000×g at 4° C. and the supernatant discarded. The beads are washed three times with RIPA buffer containing EDTA. The immunoprecipitate is eluted from the beads using 2 mM EDTA and 0.2 M glycine, pH 2.6 and immediately neutralized by the equal volume addition of Tris, pH 8.0. The eluate is collected and transferred to another microcentrifuge tube after pelleting the beads by centrifugation at 4° C. and 3,000×g.

A portion of the eluate or nuclease-free water (2 μL) is incubated at 32° C. for 5 minutes with 50 nM of self-cleaving hemagglutinin aptamer in a buffer containing 50 mM Tris pH 8.0 and 25 mM sodium chloride in a final volume of 9 μL. Magnesium chloride (1 μL) is added to the reaction at a final concentration of 8 mM and incubated for 30 minutes at 32° C. A one-step RT-qPCR master mix (15 μL) containing primers specific for the self-cleaving hemagglutinin aptamer, Superscript® III Reverse Transcriptase (Life Technologies, Grand Island, N.Y.), Amplitaq Gold® DNA Polymerase (Life Technologies, Grand Island, N.Y.), dNTPs, reaction buffer, SYBR® Green I (Life Technologies, Grand Island, N.Y.), and Rox™ (Life Technologies, Grand Island, N.Y.) is mixed with the aptamer/eluate reactions. One-step RT-qPCR is performed and monitored on a standard qPCR thermocycler. The eluates corresponding to immunoprecipitations conducted with the monoclonal antibodies against TG2 produce RT-qPCR signal above the nuclease-free water control (NWC) background reactions confirming the presence of hemagglutinin and therefore HA-PAT-2. In contrast, the RT-qPCR signal for the control mouse IgG immunoprecipitations is not above the NWC background noise confirming that the co-immunoprecipitation of HA-PAI-2 by TG2 is specific. Aptamer incubations with lysates followed by RT-qPCR confirm that both HA-PAI-2 and TG2 are present in equal concentrations in the lysates used for immunoprecipitations conducted with mouse monoclonal antibodies against TG2 and mouse IgG.

### Example 2

### Comparison of Protein Expression in Malignant and Non-Malignant Tissue Culture Cells

For the purpose of Example 2, a type I hammerhead ribozyme containing a self-cleaving K-ras-specific aptamer in the loop of stein III is used.

In order to quantify differences in expression levels of wild-type K-ms in the human oral squamous cell carcinoma line, FaDu, and in a nonmalignant primary keratinocyte line, cells are cultured, counted, and then harvested at 5×10⁶ cells per sample. A lysis buffer formulated to preserve the intended analyte, as well as the binding affinity and integrity of the self-cleaving aptamer is used to prepare whole cell lysates. RIPA buffer containing 2 millimolar EDTA is one of various lysis buffers suitable for this crude lysate preparation. Briefly, the cells are resuspended in RIPA buffer containing EDTA and gently agitated at 4° C. for 15 minutes. The lysates are subsequently cleared of cellular debris by centrifuging 10 minutes at 14,000×g at 4° C., Total protein concentrations are obtained by a standard BCA assay.

Self-cleaving K-ras aptamer (50 mM) or self-cleaving β-actin aptamer (50 mM, control) is incubated with 1 μg of cell lysates at 32° C. for 5 minutes in a buffer containing 50 mM Tris pH 8.0 and 25 mM sodium chloride in a final volume of 9 μl. Magnesium chloride (1 μL) is added to the reaction at a final concentration of 8 mM and incubated for 30 minutes at 32° C. A one-step RT-qPCR master mix (15 μL) containing primers specific for the self-cleaving K-ms aptamer, SuperScript® HI Reverse Transcriptase (Life Technologies, Grand Island, N.Y.), Ampiitaq Gold® DNA Polymerase (Life Technologies, Grand Island, N.Y.), dNTPs, reaction buffer, SYBR® Green 1 (Life Technologies, Grand Island, N.Y.), and Rox™ (Life Technologies, Grand Island, N.Y.) is mixed with the aptamer/lysate reactions. One-step RT-qPCR is performed and monitored on a standard qPCR thermocycler.

The lysate corresponding to the FaDu cell line exhibits a K-ras-dependent RT-qPCR signal greater than that for primary keratinocytes. These data correspond well with the observation that elevated K-ras is present in squamous cell carcinoma lines. The β-actin-dependent RT-qPCR signal for lysates of both cells lines is equal, demonstrating equal amounts of protein in the assay.

### Example 3

### Method for Detecting an Apoptotic Signaling Pathway

For the purpose of Example 3, type 1 hammerhead ribozymes containing self-cleaving caspase-specific (i.e., Caspase-3 and Caspase-8) and β-actin-specific (control) aptamers, each of which is located in the loop of stem III, are used.

TNF treated (25 ng/mL) cells are grown in the presence or absence of CHX, a protein synthesis inhibitor cycloheximide which enhances apoptosis, for 0, 2, 5 or 7 hours. Whole cell extracts are obtained by lysing the cells in 50 mM Tris-HCl, pH 7.6, 250 mM NaCl, 1% Triton X-100, 0.5% Nonident P-40, 3 mM EDTA, 3 mM EGTA, 10% glycerol, 2 mM DTT, 1 mM PMSF, 1 mM sodium orthovanadate, and a protease inhibitor cocktail.

A portion of the extracts or nuclease-free water (2 μL) is incubated at 32° C. for 5 minutes with 50 nM of self-cleaving p43/41 and p20 (Caspase-8) or p32 and p17 (Caspase-3) aptamers in a buffer containing 50 mM Tris pH 8.0 and 25 mM sodium chloride in a final volume of 9 Additionally, similar extracts are subjected to self-cleaving β-actin aptamers, which serve as a control. Magnesium chloride (1 μL) is added to the reaction at a final concentration of 8 mM and incubated for 30 minutes at 32° C. A one-step RT-qPCR master mix (15 μL) containing primers specific for the self-cleaving caspase-8, caspase-3 or β-actin aptamer, Amplitaq Gold® DNA Polymerase (Life Technologies, Grand Island, N.Y.), dNTPs, reaction buffer, SYBR® Green I (Life Technologies, Grand Island, N.Y.), and Rox™ (Life Technologies, Grand Island, N.Y.) is mixed with the aptamer/eluate reactions. One-step RT-qPCR is performed and monitored on a standard qPCR thermocycler.

The MEF extracts probed with the self-cleaving Caspase-8 aptamers produce an RT-qPCR signal above the nuclease-free water control (NWC) background reactions in a time-dependent manner as a function of increasing Caspase-8 detection. Extracts of TBK1 deficient MEFs consistently produce more signal at each time-point over wild-type MEFs. Additionally, TBK1 deficient MEFs produce a similar time-dependent increase in RT-qPCR signal above the NWC. Probing all samples with self-cleaving β-actin aptamers provides the sample control, as all MEF extracts produced a similar RT-qPCR signal above the NWC samples using this aptamer. This confirms that TBK1 deficient MEF extracts are more sensitive to TNF-induced apoptosis, which is associated with elevated caspase-8 and caspase-3 activation.

It will be appreciated by those skilled in the art that changes could be made to the embodiments described above without departing from the broad inventive concept thereof. In some instances, for example, it will be appreciated that the steps comprising the method 10 shall be performed sequentially or in the order in which they are illustrated in FIG. 1. It is understood, therefore, that the present disclosure is not limited to the particular embodiments disclosed, but it is intended to cover modifications that are within the spirit and scope of the present disclosure. All patents, publications, and references disclosed herein are incorporated by reference in their entirety.

What is claimed is:

1. A method for determining the amount of a target analyte in a sample, the method comprising (a) contacting the sample with a self-cleaving synthetic molecule to form a first treated sample, the self-cleaving synthetic molecule including at least one target analyte-specific aptamer, (b) contacting the first treated sample with at least one actuating molecule to form a second treated sample, and (c) subjecting the second treated sample to a target amplification assay, wherein a detectable signal is produced by the target amplification assay when the target analyte is present in the sample.

2. The method of claim 1 wherein the sample is contacted with at least one chelating agent prior to step (a).

3. The method of claim 1 wherein the target analyte is selected from the group consisting of small molecules, polypeptides, nucleic acids, carbohydrates, fatty acids, cells, tissues, microorganisms, and metabolic precursors or products thereof.

4. The method of claim 1 wherein the target analyte is not isolated from the sample prior to steps (a)-(c).

5. The method of claim 1 wherein the at least one chelating agent is ethylenediaminetetraacetic acid (EDTA).

6. The method of claim 1 wherein step (a) is performed in a low salt environment at a temperature of about 4° C. to about 32° C.

7. The method of claim 2 further including contacting the sample with one or more non-specific nucleic acids.

8. The method of claim 1 wherein the self-cleaving synthetic molecule is a type I hammerhead ribozyme comprising a catalytic core and stem I, stem II, and stem III duplex regions extending therefrom, each of stem I, stem II, and stem III having a single-stranded loop region.

9. The method of claim 8 wherein the loop region of stem III includes the at least one target analyte-specific aptamer.

10. The method of claim 8 wherein the type I hammerhead ribozyme includes first and second primer sites at the 3′ and 5′ ends thereof, respectively.

11. The method of claim 1 wherein the at least one actuating molecule is contacted with the second sample in an amount sufficient to cause self-cleavage of the self-cleaving synthetic molecule in the absence of the target analyte.

12. The method of claim 11 wherein the at least one actuating molecule is a cation.

13. The method of claim 10 wherein the 3′ primer site is self-cleaved from the type hammerhead ribozyme in the absence of the target analyte.

14. The method of claim 10 wherein the 3′ primer site is not cleaved from the type I hammerhead ribozyme in the presence of the target analyte.

15. The method of claim 1 wherein the target amplification assay is quantitative polymerase chain reaction (qPCR).

16. The method of claim 15 wherein the qPCR is reverse transcriptase-qPCR (RT-qPCR).

17. The method of claim 11 wherein the uncleaved target analyte-specific aptamer is amplified by the target amplification assay.

18. The method of claim 1 being performed in less than about 2 hrs.

19. A method for determining the amount of a target polypeptide in a sample, the method comprising (a) contacting the sample with a type I hammerhead ribozyme to form a first treated sample, the type I hammerhead ribozyme including at least one target analyte-specific aptamer and first and second primer sites at the 3′ and 5′ ends thereof, respectively, (b) contacting the first treated sample with at least one actuating molecule to form a second treated sample, and (c) subjecting the second treated sample to RT-qPCR, wherein the 3′ primer site is not cleaved from the type I hammerhead ribozyme in the presence of the target analyte and wherein an uncleaved target analyte-specific aptamer is amplified by the target amplification assay to produce a detectable signal.

20. A kit for determining the amount of a target analyte in a sample, the kit comprising (a) at least one test tube for preparing a sample for a target amplification reaction, (b) reagents for conducting the method of claim 1, and (c) instructions for detecting the target analyte according to claim 1.


SEQUENCE LISTING (6 sequences)

SEQ ID NO: 1 - RNA, 69 nt, Artificial Sequence
  Description of Artificial Sequence Synthetic oligonucleotide
cucucgcucacugaagaaggcannnnnnugccugaaagggannnnucccuaugagccuucccucgcaca

SEQ ID NO: 2 - RNA, 18 nt, Artificial Sequence
  Description of Artificial Sequence Synthetic oligonucleotide
ugagccuucccucgcaca

SEQ ID NO: 3 - RNA, 51 nt, Artificial Sequence
  Description of Artificial Sequence Synthetic oligonucleotide
cucucgcucacugaagaaggcannnnnnugccugaaagggannnnucccua

SEQ ID NO: 4 - RNA, 43 nt, Artificial Sequence
  Description of Artificial Sequence Synthetic oligonucleotide
cucucgcucacugaagaaggcanugccugaaaggganucccua

SEQ ID NO: 5 - DNA, 18 bp, Artificial Sequence
  Description of Artificial Sequence Synthetic primer
tgtgcgagggaaggctca

SEQ ID NO: 6 - RNA, 61 nt, Artificial Sequence
  Description of Artificial Sequence Synthetic oligonucleotide
cucucgcucacugaagaaggcanugccugaaaggganucccuaugagccuucccucgcaca